(19) United States
(12) Patent Application Publication
(10) Pub. No.: US 2006/0259148 A1
(43) Pub. Date: Nov. 16, 2006

(54) Cartilage-preserving long bone head prosthesis

(75) Inventor: Yaacov Bar-Ziv, Raanana (IL)

Correspondence Address: NIXON & VANDERHYE, PC, 901 NORTH GLEBE ROAD, 11TH FLOOR, ARLINGTON, VA, 22203, US

(73) Assignee: PreSRV Ltd., Yokneam (IL)

(21) Appl. No.: 11/411,244

(22) Filed: Apr. 26, 2006

Related U.S. Application Data
(63) Continuation-in-part of application No. PCT/IL04/00980, filed on Oct. 27, 2004.

(30) Foreign Application Priority Data
Oct. 28, 2003 (IL) 158633
Dec. 14, 2005 (IL) 172588

(51) Int. Cl. A61F 2/36; A61F 2/40; A61F 2/30

(52) U.S. Cl. 623/19.14; 623/23.11; 623/23.42; 623/908

(57) ABSTRACT

The present invention is primarily directed to a long-bone endoprosthesis comprising an essentially spherical head region that is connected to a narrowed stem section, wherein the head region is characterized in having a roughened outer surface. The invention also provides methods for treating fractures of the neck region of a long-bone in a patient in need of such treatment, wherein said methods are characterized by the preservation of a long-bone head shell.

### FIELD OF THE INVENTION

The present invention relates to a new type of prosthetic device, and a method that uses said device in the treatment of femoral and humeral neck fractures. More specifically, the prosthetic device of the invention is capable of being used to treat long bone neck fractures in procedures wherein the natural articular cartilage and subchondral bone of the long bone is preserved.

### BACKGROUND OF THE INVENTION

Fractures of the neck of the femur and humerus have always presented great challenges to orthopedic surgeons and remain in many ways today the “unsolved fracture” as far as methods of treatment and the results obtained thereby are concerned.

Approximately 280,000 hip fractures occurred in the United States in 1998. The National Osteoporosis Foundation reported that in 1995, health care expenditures for the management of osteoporotic hip fractures totaled $8.7 billion, representing 63% of the cost of treating all osteoporotic fractures, and 43% of the cost of all fracture care. It is predicted that by the year 2020, the incidence of hip fractures in the United States will be more than 500,000 new cases per year. It is further predicted that the cost of treating these patients will rise to $16 billion per year.

High rates of avascular necrosis and nonunion are common complications in displaced fractures of the femoral neck (Garden classification stages 3 and 4). Even when undisplaced, there is no assurance that a fracture of the femoral neck may be treated in a satisfactory way. One of the key reasons for the problematic nature of these fractures from the therapeutic aspect is that the surgeon has less control over avascular necrosis, because of the disturbances to blood flow to the femoral head that occur following femoral neck fracture.

Femoral neck fractures are usually entirely intracapsular, and (in common with all intracapsular fractures) the synovial fluid bathing the fracture may interfere with the healing process. Furthermore, all healing must be take place endo-osteally, in view of the fact that the femoral neck has essentially no periosteal layer. Finally, angiogenic-inhibiting factors in synovial fluid can also inhibit fracture repair. All of these factors, together with the aforementioned precarious blood supply to the femoral head, result in unpredictable healing and a subsequent fairly high incidence of nonunions.

Few treatment options are available for the management of long bone neck fractures. Of these options, the most commonly employed are:

1. Osteosynthesis—fracture fixation; and

2. Hemiartheoplasty—replacement of the femoral head with a (normally metal) prosthetic head attached to a monoblock or modular femoral stem. The most popular types of prosthesis used are the Thompson, Austin-Moore, bipolar and unipolar prostheses. A typical bipolar femoral prosthesis is shown in FIG. 1.

Many surgeons recommend the second option, that is, the use of a primary prosthetic replacement for use in the treatment of elderly by ambulatory patients. While the use of prostheses may assist in the prevention of nonunion and avascular necrosis, their use is associated with a number of other complications.

One of the recognized disadvantages of using a prosthesis in the management of a fresh femoral neck fracture is the pain that is produced as a consequence of acetabular erosion. This complication is often so severe that it has prompted one expert to comment:
“The sacrifice of the head and neck and replacement by a metallic foreign substance is not the answer for the majority of patients; in over half, the best available material is in the acetabulum, and its indiscriminate removal should be avoided.” (Salvatore “Campbell's Operative Orthopedics, 9^{th} edition.)

A further major problem associated with the use of existing prosthetic devices is the dysfunction that arises from the mismatch between the acetabulum and the prosthetic head. Further traumatic complications also arise from the fact that following implantation of the prosthesis, articulation takes place between the hard metal of the prosthetic head and the much softer acetabular surface.

It is a purpose of the present invention to provide an improved prosthetic device that may be used in the treatment of long bone neck fractures.

It is another purpose of the invention to provide a prosthetic device whose use in the treatment of long bone neck fractures does not require the loss of either of the two natural articulating surfaces of the proximal long bone joint.

It is yet another purpose of the invention to provide a method of treatment of long bone fractures that allows the use of an endoprosthesis while preserving the natural articular surfaces.

It is a further purpose of the invention to provide a prosthetic device and method that may be used to overcome the disadvantages and problems associated with prior art devices and methods.

Further objects and advantages of the present invention will become apparent as the description proceeds.

### SUMMARY OF THE INVENTION

It has now been unexpectedly found that it is possible to treat fractures of the neck region of the femur and humerus by means of a surgical method involving the use of a novel endoprosthesis. The head region of this endoprosthesis is characterized by being adapted for insertion into a shell-like cavity comprising the outer layers of the patient's femoral or humeral head, following removal of most of the cancellous bone. In this way, the cartilaginous articular surface of the long bone head, together with the underlying subchondral bone is preserved, thus retaining the natural articulating surfaces of the proximal long bone ball-and-socket joint.

The present invention is thus primarily directed to a long-bone endoprosthesis comprising an essentially spherical head region that is connected to a narrowed stem section, wherein the head region is characterized in having a roughened outer surface.

In one preferred embodiment of the endoprothesis of the present invention, the roughened prosthetic head outer surface has an arithmetic average roughness (R_{α}) value in the range of 0.05 μm to 500 μm. More preferably, the R_{α} value is between 40 μm and 200 μm. Most preferably, the R_{α} value is 50 μm. For the purposes of the present invention, the parameter R_{α} is defined in accordance with the International Standards Organization document ISO 468 (“Surface Roughness Parameters—their values and general rules for specifying requirements”).

In another preferred embodiment, the roughened prosthetic head outer surface is a surface comprising one or more surface features selected from the group consisting of indentations, ridges, slots, grooves, pores, dimples and protuberances. Theses features may be introduced into the surface of the prosthetic head by any standard procedure including mold-casting techniques, machine-cutting and (in the case of small-diameter surface protuberances) grit blasting techniques. In a particularly preferred embodiment, the roughened prosthetic head outer surface is a surface fitted with one or more grooves or slots, as will be described in more detail hereinbelow.

In the context of the present invention, the term “long-bone” is used primarily to refer to the femur and humerus.

In one preferred embodiment of the prosthesis of the invention, the head and neck regions of the prosthesis are constructed as a one-piece unit, to be referred to hereinafter as the monoblock embodiment.

In another preferred embodiment, the head and neck regions are constructed as two separate modular units. In a particularly preferred embodiment of this aspect of the invention, the separate head region is a bipolar prosthetic head.

In another aspect, the present invention is directed to a long-bone endoprosthetic head comprising an essentially spherical portion, into the distal side of which is formed a recess such that a femoral stem endoprosthesis may be inserted therein and connected thereto, and wherein said endoprosthetic head is characterized by having a roughened outer surface, as defined and described hereinabove.

Thus, in one preferred embodiment of the aforementioned endoprosthetic head of the present invention, the roughened prosthetic head outer surface has an arithmetic average roughness (R_{α}) value in the range of 0.05 μm to 500 μm. More preferably, the R_{α} value is between 40 μm and 200 μm. Most preferably, the R_{α} value is 50 μm.

In another preferred embodiment, the roughened prosthetic head outer surface is a surface comprising one or more surface features selected from the group consisting of indentations, ridges, slots, grooves, pores, dimples and protuberances. In one preferred embodiment of this aspect of the invention, the average depth or height of the aforementioned surface features is in the range of 0.05 μm to 5000 μm. More preferably, the average value is between 400 μm and 2000 μm. Most preferably, the average height or depth value is 1000 μm. In another preferred embodiment, in the case of slots, grooves and pores, the depth of said features is in the range of 1 μm to the maximum thickness of the material of the prosthetic head surface. In the case of this maximum depth value, the prosthetic head is actually perforated by the slots, grooves or pores, such that a channel is created from the external surface of the prosthetic head to the inner surface or cavity of said head.

In a further preferred embodiment of the invention, the aforementioned long-bone endoprosthetic head having a roughened outer surface is constructed as a bipolar prosthetic head, having an outer head (with the roughened outer surface), an intermediate portion adjacent to the inner surface of said outer head, and an inner head that articulates with the inner surface of said intermediate portion.

The present invention is further directed to a long-bone endoprosthesis comprising an essentially spherical head comprising a central internal cavity that perforates one side of the prosthetic head, such that said cavity is suitable for articulation with a long-bone prosthetic stem section, and wherein said head region is characterized in having crater-like depressions distributed over its outer surface, and wherein substantially the entire outer surface has an arithmetic average micro-roughness (R_{α}) in the range of 1-1000 micrometers. Preferably, the average micro-roughness value should be between 30 and 70 micrometers.

In one preferred embodiment of this aspect of the invention, the crater-like depressions are distributed across essentially the entire outer surface of the endoprosthetic head, each depression having a maximum depth in the range of 0.2 to 3 mm and a diameter in the range of 3 to 20 mm.

The term “micro-roughness” is used herein, in the context of this embodiment of the invention, to indicate roughness that is due to small irregularities of the surface. This type of roughness is clearly different from the discontinuities in the smooth, essentially spherical envelope shape of the prosthetic head that are due to features such as the craters found on the outer surface of the presently-disclosed device.

As mentioned hereinabove, the parameter R_{α} is defined in accordance with the International Standards Organization document ISO 468 (“Surface Roughness Parameters—their values and general rules for specifying requirements”).

The present invention also encompasses an endoprosthetic system comprising a long-bone endoprosthetic head as disclosed and defined hereinabove, and a femoral stem endoprosthesis that may be connected thereto.

In a further aspect, the present invention is also directed to a method for treating fractures of the neck region of a long-bone in a patient in need of such treatment, wherein said method comprises the steps of:

a) removing most or all of the cancellous bone from the head of said long-bone, thereby forming a long-bone head shell;

b) preparing the long bone canal to accept a prosthetic stem;

c) inserting, and optionally cementing said prosthetic stem into said long-bone canal;

d) inserting and cementing a long-bone prosthetic head into said long-bone head shell; and

e) reduction of the stem region into the recessed region of said prosthetic head.

The present invention is also directed to a method for treating fractures of the neck region of a long-bone in a patient in need of such treatment, wherein said method comprises the steps of:

a) removing the native long-bone head from its natural location in the body, by the use of a safe technique that does not cause injury to the cartilage or subchondral bone;

b) preparing the head shell outside the body on the operative tray;

c) preparing the long bone canal to accept a prosthetic stem section;

d) inserting the prosthetic stem section with or without the use of cement;

e) cementing the prosthetic head section to the shell formed in step (b);

f) reduction of the prepared prosthetic head together with the attached head shell into the joint of the patient being treated, and connection of the distal portion of said prosthetic head to said prosthetic stem section.

The present invention is further directed to a method for treating fractures of the neck region of a long-bone in a patient in need of such treatment, wherein said method comprises the steps of:

a) removing the native long-bone head from its natural location in the body, by the use of a safe technique that does not cause injury to the cartilage or subchondral bone;

b) preparing the head shell outside the body on the operative tray;

c) preparing the long bone canal to accept the stem section of a monoblock prosthesis;

d) inserting said monoblock prosthesis stem section into said long bone canal with or without the use of cement;

e) cementing the head section of said monoblock prosthesis to the shell formed in step (b);

f) reduction of the prepared monoblock prosthesis head together with the attached head shell into the joint of the patient being treated.

The present invention is also directed to another method for treating fractures of the neck region of a long-bone in a patient in need of such treatment, wherein said method comprises the steps of:

a) removing most or all of the cancellous bone from the head of said long-bone, thereby forming a long-bone head shell;

b) preparing the long bone canal to accept a prosthetic stem;

c) inserting, and optionally cementing said prosthetic stem into said long-bone canal;

d) cementing the outer head of a bipolar prosthesis to the shell formed in step (a);

e) placing prosthetic stem section trunion into the inner cavity of the inner prosthetic head;

f) inserting the inner prosthetic head into the inner cavity of the outer prosthetic head;

g) locking the bipolar head assembly by means of closing the locking ring.

The present invention is further directed to a method for treating fractures of the neck region of a long-bone in a patient in need of such treatment, wherein said method comprises the steps of:

a) removing the native long-bone head from its natural location in the body, by the use of a safe technique that does not cause injury to the cartilage or subchondral bone;

b) preparing the head shell outside the body on the operative tray;

c) preparing the long bone canal to accept a prosthetic stem section;

d) inserting the prosthetic stem section with or without the use of cement;

e) cementing the outer head of a bipolar prosthesis to the shell formed in step (b);

f) placing prosthetic stem section trunion into the inner cavity of the inner prosthetic head;

g) inserting the inner prosthetic head into the inner cavity of the outer prosthetic head;

h) locking the bipolar head assembly by means of closing the locking ring;

i) reduction of the prepared prosthetic head together with the attached head shell into the joint of the patient being treated.

The present invention is further directed to another method for treating fractures of the neck region of a long-bone in a patient in need of such treatment, wherein said method comprises the steps of:

a) removing the native long-bone head from its natural location in the body, by the use of a safe technique that does not cause injury to the cartilage or subchondral bone;

b) preparing the head shell outside the body on the operative tray;

c) preparing the long bone canal to accept the stem-inner section of a monoblock bipolar prosthesis;

d) inserting the prosthetic stem section of the stem-inner head bipolar monoblock bipolar prosthesis, with or without the use of cement;

e) cementing the bipolar prosthetic outer head section to the shell formed in step (b);

f) reduction of the prepared bipolar prosthetic outer head together with the attached head shell into the joint of the patient being treated;

g) inserting the bipolar inner head into the internal cavity of the bipolar outer head;

h) locking the bipolar head assembly by means of closing the locking ring.

The present invention is further directed to yet another method for treating fractures of the neck region of a long-bone in a patient in need of such treatment, wherein said method comprises the steps of:

a) removing the native long-bone head from its natural location in the body, by the use of a safe technique that does not cause injury to the cartilage or subchondral bone;

b) preparing the head shell outside the body on the operative tray;

c) preparing the long bone canal to accept the stem-inner section of a monoblock bipolar prosthesis;

d) inserting the prosthetic stem section of the stem-inner head bipolar monoblock bipolar prosthesis, with or without the use of cement;

e) inserting the bipolar inner head into the internal cavity of the bipolar outer head;

f) locking the bipolar head assembly by means of closing the locking ring;

g) cementing the shell formed in step (b) onto the bipolar prosthetic outer head section;

h) reduction of the prepared bipolar prosthetic outer head together with the attached bipolar prosthesis into the joint of the patient being treated.

The present invention is also directed to a yet further method for treating fractures of the neck region of a long-bone in a patient in need of such treatment, wherein said method comprises the steps of:

a) removing most or all of the cancellous bone from the head of said long-bone, thereby forming a long-bone head shell;

b) preparing the long bone canal to accept the stem-inner head section of a monoblock bipolar prosthesis;

c) inserting, the prosthetic stem section of the stem-inner head bipolar monoblock bipolar prosthesis, with or without the use of cement;

d) inserting the bipolar inner head into the internal cavity of the bipolar outer head;

e) locking the bipolar head assembly by means of closing the locking ring;

f) cementing the bipolar prosthetic outer head section to the shell formed in step (b).

The above methods of treatment are applicable to the treatment of fractures of both the femur and the humerus.

All the above and other characteristics and advantages of the present invention will be further understood from the following illustrative and non-limitative examples of preferred embodiments thereof.

### BRIEF DESCRIPTION OF THE DRAWINGS

FIG. 1 is a photograph of a prior art bipolar femoral endoprosthesis.

FIG. 2 schematically depicts the prosthetic device of the present invention.

FIG. 3 is a photographic representation of a prosthetic device of the present invention after insertion of the head of the device into the shell of a femoral head, following removal of most of the cancellous bone.

FIG. 4 is a photograph demonstrating the head of a two-part prosthetic device according to the present invention following its insertion into a femoral head shell.

FIG. 5 is a side view of one embodiment of a femoral endoprosthetic head according to the present invention, in which the roughened surface is provided by the presence of both circumferential and longitudinal slots.

FIG. 6 is an inferior view of the embodiment of the endoprosthetic head depicted in FIG. 5.

FIG. 7 is a superior view of the embodiment of the endoprosthetic head depicted in FIGS. 5 and 6, showing the non-slotted region at the proximal pole of said head.

FIG. 8 is an inferior view of a bipolar prosthetic head depicting the inner head portion residing within the internal cavity of the outer head portion.

FIG. 9 is an inferior view of a bipolar prosthetic head depicting the inner head locked in place in the internal cavity of the outer head portion by means of a locking ring.

FIG. 10 photographically depicts cratered, micro-roughened endoprosthetic head of the present invention.

FIG. 11 schematically depicts the manner in which the endoprosthetic head of the present invention is assembled together with cement, a thin cancellous bone layer and the outer cortical bone/cartilage shell. FIG. 11A shows the order of assembly of the aforementioned layers and components, while fig. 11B depicts the completed assembly.

FIG. 12 is a cut-away illustration of a femoral endoprosthetic head of the present invention in situ, i.e. in articulation with the acetabular cavity.

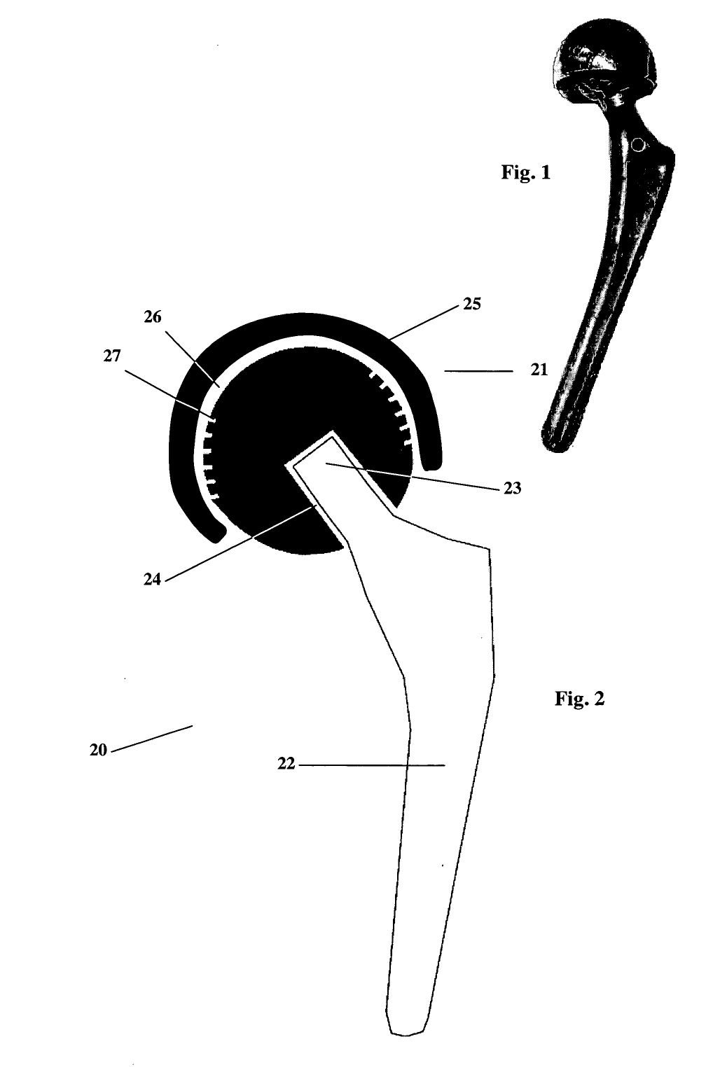

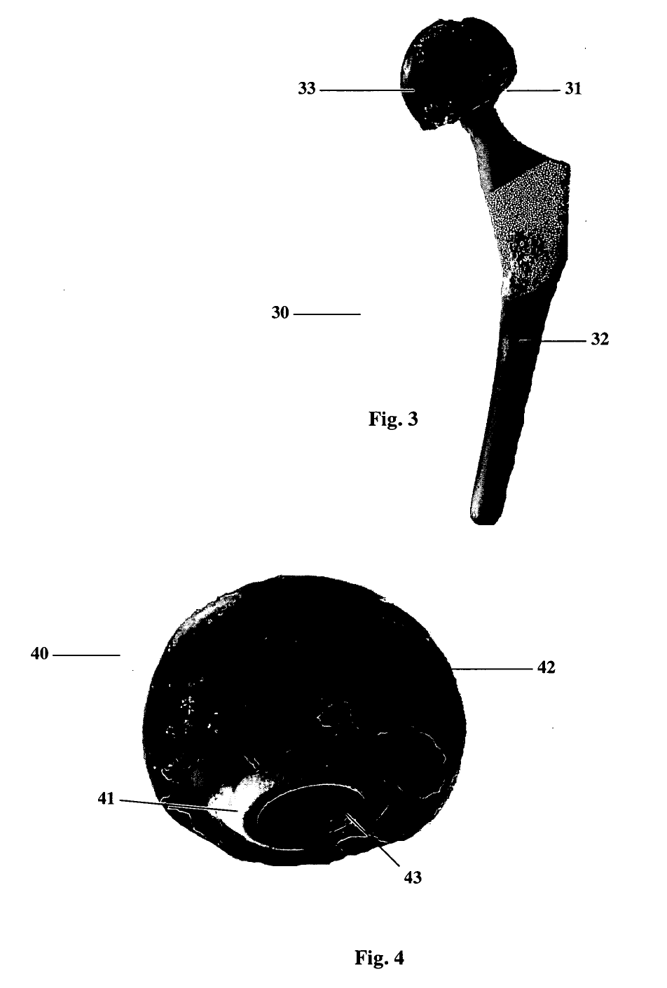

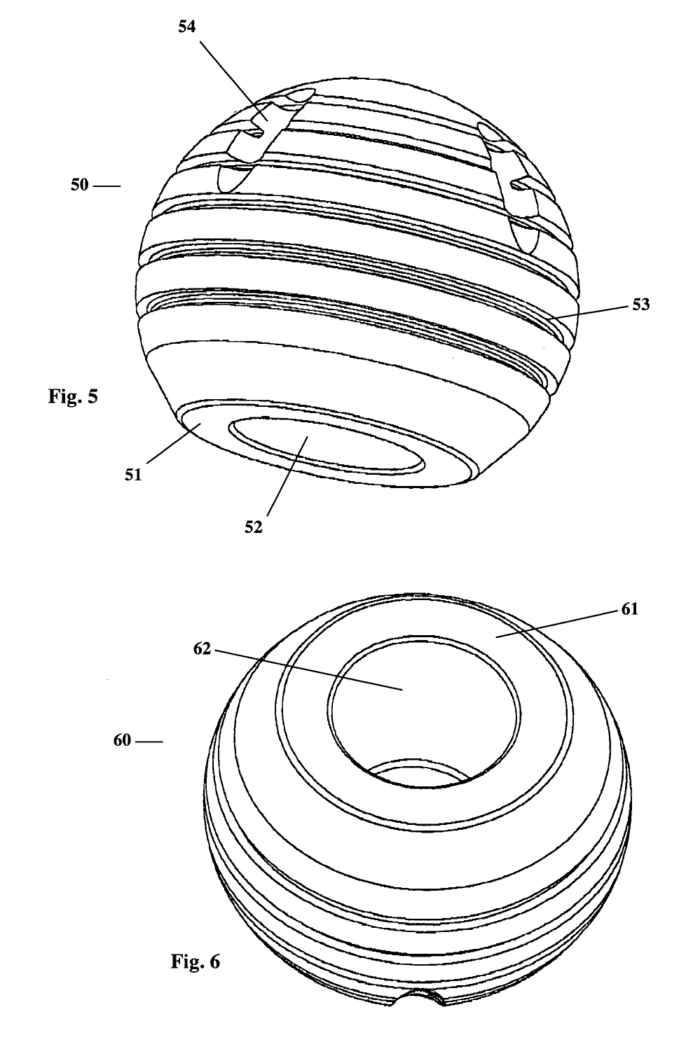

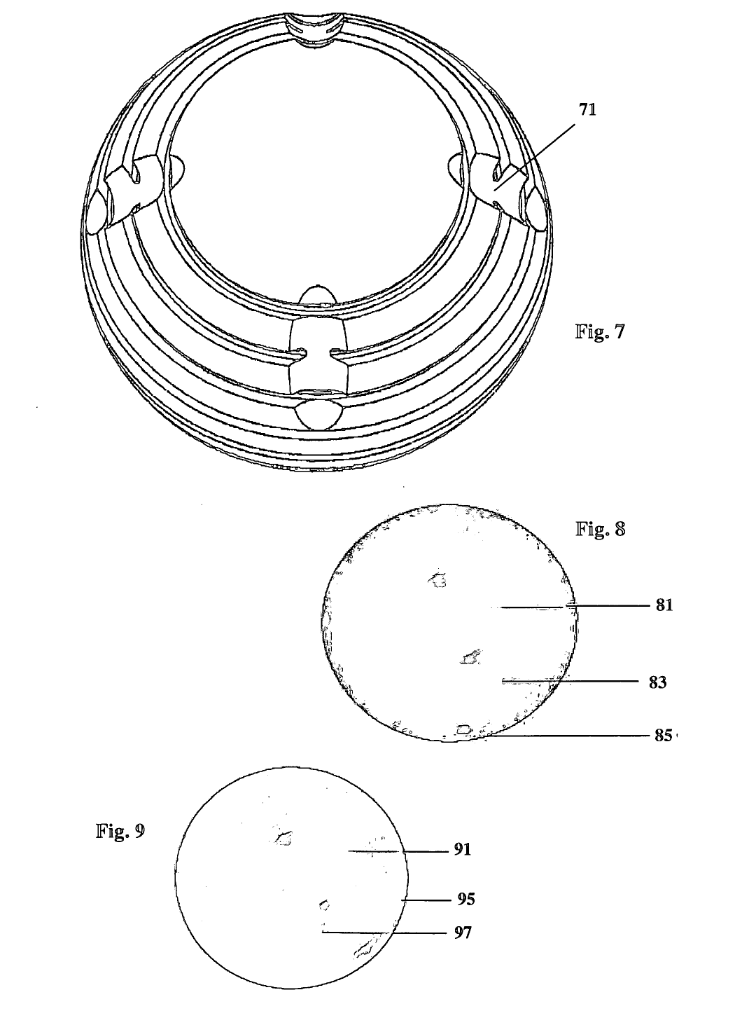

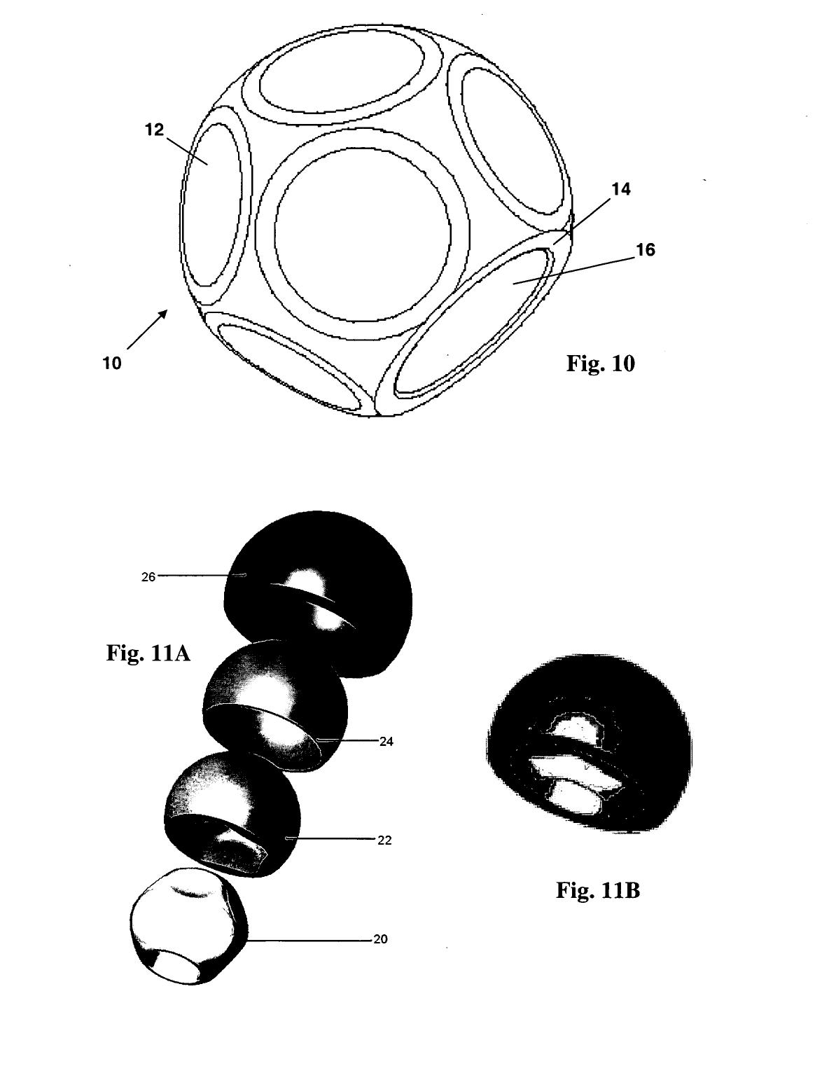

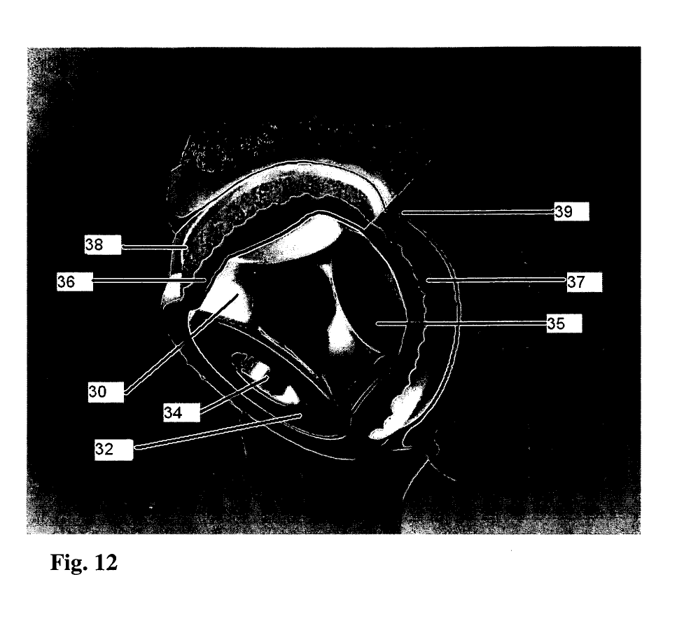

### DETAILED DESCRIPTION OF THE PREFERRED EMBODIMENTS

The essential inventive feature of the endoprosthesis of the invention is the fact that the head region (or at least a portion thereof) has a roughened, non-polished outer surface, in order to permit optimal mechanical bonding of said head region to cement, and by which means to the inner surface of the femoral or humeral head shell-like cavity. This stands in marked contrast to prior art long-bone head and stem prostheses, whose head regions are characterized by having a polished surface, the purpose of which is to replace the natural cartilaginous articular surface of the natural femoral or humeral head. The retention of the natural articular surfaces of the ball-and-socket joints results in several clinical advantages, including prevention of acetabular erosion, obviation of the problem of ball/socket mismatch that is seen with prior art approaches, and pain reduction. Many of the above advantages arise from the fact that the retention of the anatomical articulating surfaces preserves the natural clearance between the long bone head and the socket within the joint. It should be noted that the viability and integrity of the long bone head articular cartilage is maintained by virtue of the fact that the nutritional requirements of this tissue are met by synovial fluid that bathes said tissue.

A typical femoral endoprosthesis in accordance with the present invention is schematically illustrated in FIG. 2. The prosthesis, shown generally as 20 consists essentially of two sections: the prosthetic head region 21 and the stem region 22. In the lateral view shown in this figure, it may be seen that the proximal end 23 of the stem region is inserted into an elongated recess 24 in said head region. The femoral head shell 25, comprising the patient's articular cartilage and associated subchondral bone, is bonded to the prosthetic head region 21 by means of biocompatible cement 26. The surface roughness of the prosthetic head region 21 of the embodiment depicted in this figure is provided by a series of circumferentially arranged slots 27. As indicated in the figure, the biocompatible cement 26 is able to penetrate into said slots, thus increasing the strength of the bonding between the prosthetic head region 21 and the femoral head shell 25.

FIG. 3 photographically depicts a prosthesis of the present invention, generally shown as 30 after assembly of the head 31 and stem 32 regions. Most of the surface area of head region 31 is obscured by the femoral head shell 33 that covers said region.

FIG. 4 photographically depicts the head region of the prosthesis shown in FIG. 3 (generally shown here as 40) prior to insertion of the stem region. A small portion of the roughened outer surface 41 of the head region is shown extending below the inferior (distal) margin of the femoral head shell 42. It will be noted that said surface is perforated by a circular opening 43 into which the stem region of the prosthesis will be inserted.

In another particularly preferred embodiment of the invention, the long-bone endoprosthetic head is characterized by the presence of a plurality of facets or craters distributed over its outer surface. In addition, essentially the entire outer surface of the prosthetic head is micro-roughened. It was unexpectedly found that the combination of these two surface features (i.e. the cratering and the micro-roughness) significantly improves the mechanical stability of the attachment between the prosthetic head and the cartilage/bone shell that is cemented thereonto.

The dimensions of the internal cavity of the prosthetic head are such that a standard long-bone prosthetic stem may be fitted therein. In practice, this generally requires that the diameter of opening 16 be in the range of 18 to 38 mm, preferably 10 to 15 mm.

The aforementioned craters have a diameter in the range of 3 to 20 mm, preferably 12.5 mm, and a maximum depth in the range of 0.2 to 3 mm. A typical prosthetic head having an external diameter of 28 mm will have approximately 11 facets on its outer surface. These craters are created by one or more of the following means: electro-erosion, milling, casting or sintering.

Following creation of the craters or facets, as described hereinabove, the outer surface of the prosthetic head is micro-roughened. This micro-roughening may be produced in several different ways, by the use of any standard procedure including mold-casting techniques, machine-cutting, electro-erosion, sintering and grit blasting techniques.

This embodiment of the endoprosthesis of the present invention may be constructed from cobalt-based alloys (e.g. cobalt-chrome), titanium, titanium-based alloys, stainless-steel, combinations of the above-mentioned metals, and other metals and metal combinations used in orthopedic implants. The prostheses are manufactured from the aforementioned metals by means of one or more of: electro-erosion, casting, sintering, milling and turning.

In addition, the endoprostheses may also comprise components that are constructed from non-metallic materials such as biocompatible plastics and polymers, such as polyurethane and polyethylene, as well as other synthetic biocompatible materials that are softer than the aforementioned metals, and hard materials such as ceramics. The aforementioned lists are intended to exemplify some of the more common materials, and are not to be considered as limiting. Various combinations of the different materials mentioned hereinabove also form part of the scope of the present invention. Thus, for example, prosthetic heads may be constructed with a metal trunion in contact with a plastic body. Another example of the use of a combination of materials would be the case in which the central bulk of the prosthetic head is constructed of a metal or metal alloy, whereas the outer portion (having an external surface that is cemented to the femoral head shell and an inner surface that is bonded to said metal or alloy) may be constructed of a non-metallic, polymeric material.

In a preferred embodiment of this embodiment of the invention, the prosthetic head is manufactured from a Cobalt-Chrome alloy conforming to ASTM F1537, such as can be procured from Allvac, Monroe, N.C., USA, item number TJA-1537.

Further details of this embodiment will be shown in illustrative Example 2, hereinbelow.

In one preferred embodiment of the present invention, the roughened and/or featured prosthetic head may form part of a bipolar prosthesis. As is well known in the art, bipolar prostheses for use in the management of long bone neck fractures comprise the following components:
1. An outer head for articulation with the joint socket (e.g. acetabular) surface. In the case of the present invention, the outer head has a roughened outer surface, in order to permit attachment thereof to the prepared femoral head, as described herein.
2. An intermediate layer or portion fixed within the inner cavity of the outer head. Typically, the intermediate portion is constructed of a material that is softer than metal (such as polyethylene). Alternatively, this portion may be constructed of the same material as the outer head, thereby forming a single unit therewith. (More details of the materials used in the construction of bipolar prostheses according to the present invention are given hereinbelow.)
3. An inner head, the outer surface of which movably articulates with the inner surface of the aforementioned intermediate portion, and the inner cavity of which is immovably fixed to the femoral stem trunion. FIG. 8 illustrates the articulation of the inner head 81 with the intermediate portion 83 within the inner cavity of the outer head 85.
4. A locking ring, whose function is to retain the inner head in movable contact with the intermediate portion within the inner cavity of the outer head. FIG. 9 illustrates (from an inferior aspect) the relative disposition of the inner head 91 and outer head 95, following closure of the locking ring 97.

In another preferred embodiment of the present invention, the prosthetic head having the roughened and/or cratered or otherwise featured outer surface may be part of a bipolar monoblock prosthesis, wherein the inner head and femoral stem of said prosthesis are provided as a single integral unit. As in the case of the bipolar prosthetic head described hereinabove, the inner head of the monoblock unit is retained in movable contact with the intermediate layer within the cavity of the outer head and locked in place with the above-described locking ring.

The endoprostheses of the present invention may, in general, be constructed from cobalt-based alloys (e.g. cobalt-chrome), titanium, titanium-based alloys, stainless-steel, and combinations of the above-mentioned metals. In addition, the endoprostheses may also comprise components that are constructed from non-metallic materials such as biocompatible plastics and polymers, such as polyurethane and polyethylene, as well as other synthetic biocompatible materials that are softer than the aforementioned metals, and hard materials such as ceramics. The aforementioned lists are intended to exemplify some of the more common materials, and are not to be considered as limiting. Various combinations of the different materials mentioned hereinabove also form part of the scope of the present invention. Thus, for example, prosthetic heads may be constructed with a metal trunion in contact with a plastic body. Another example of the use of a combination of materials would be the case in which the central bulk of the prosthetic head is constructed of a metal or metal alloy, whereas the outer portion (having an external surface that is cemented to the femoral head shell and an inner surface that is bonded to said metal or alloy) may be constructed of a non-metallic, polymeric material.

In the case of a prosthetic head of the invention of a bipolar construction, the outer head (i.e. the portion bearing the roughened surface that is cemented to the femoral head shell) may be constructed of a metal or metal alloy. Alternatively, the outer head may be constructed of a ceramic or polymeric material. The intermediate layer that lines the inside of said outer head is typically constructed of polyethylene. However, in another embodiment, this layer may be constructed of the same material as the outer head, thereby forming a single unit therewith. The inner head, like the outer head, may be constructed of either a metal or of a polymeric or ceramic material.

Many different combinations of materials may be chosen in order to obtain prostheses having the desired physical properties (such as hardness, resilience, elasticity etc.).

The external dimensions of the endoprosthesis of the present invention are essentially as the same as those of prior art femoral and humeral prostheses. Thus, in the case of a prosthesis of the present invention having a head region that is essentially spherical in shape, the diameter of the spherical head is generally in the range of 22 to 40 mm. A preferred head diameter for the femoral head prosthesis is in the range of 28 to 32 mm. In practice, however, the head diameter may also be outside of these preferred ranges, in certain circumstances being as small as 12 mm or as large as 60 mm. It should be noted, however, that the prostheses of the present invention are not limited to those having head regions of spherical conformation. Rather, prostheses with non-spherical, multi-sided head conformations are also included within the scope of the presently-claimed invention. Preferably, such multi-sided head regions have three or more sides. More preferably, the number of sides present in such conformations is between four (e.g. square, rectangular and/or trapezoid) and eight (i.e. octagonal). However, multi-sided head geometries of any other type that are suitable for performing the present invention are also to be considered as falling within the scope thereof. Examples of such suitable geometries (in addition to those mentioned hereinabove) include, but are not limited to, regular pyramidal shapes as well as irregular polyhedrons, star-shaped, “hedgehog-shaped”, and so on. In addition, other head geometries such as conical, frusto-conical and variations and combinations thereof may also be usefully employed, and form part of the scope of the present invention.

It is to be emphasized that, although the external shape and dimensions of the endoprostheses of the present invention may be similar to those of corresponding prior art devices, the presently-claimed devices are distinguished by having head regions that are unpolished, and which optionally are further roughened by the presence of one or more distinctive surface features, as described hereinabove, and exemplified hereinbelow.

The roughened prosthetic head surface may be produced in several different ways, by the use of any standard procedure including mold-casting techniques, machine-cutting and (in the case of small-diameter surface protuberances) grit blasting techniques. Two particularly preferred embodiments of the prosthetic head—one having an outer surface fitted with one or more slots, and one having an outer surface which is both cratered and micro-roughened—will be described in more detail in the illustrative examples given herein below.

Many different types of cement may be used to bond the head of the prosthesis into the head “shell” formed after removal of most or all of the cancellous bone from the long bone head. Examples of suitable cements and glues include PMMA cement, Palacos cement, Simplex, CMW and Cementech.

Surgical Method:

The endoprosthesis of the present invention may be used in the surgical treatment of long-bone neck fractures. The following procedure is one example of a surgical method that utilizes the instantly-disclosed prosthesis in the management of femoral neck fractures:

1. Standard approach to the hip joint.

2. Wide exposure of the hip capsule.

3. Identification of the femoral head fractured margins.

4. Holding the femoral head with a reduction clamp or similar instrument, with the fracture plane away from the articular space.

5. Reaming the cancellous bone out of the femoral head using a high speed bur or any other conventional acetabular reamer, avoiding rotational forces in the articular space.

6. Retaining a thin layer (2-3 mm) of subchondral bone, forming (together with the femoral head articular cartilage) a femoral head “shell”.

7. Preparing the femoral canal in the standard way for a cemented or non-cemented femoral stem.

Either:

8a. Inserting cement into the femoral canal and introducing the stem into said canal. The stem is held in place within the femoral canal until polymerization of the cement is complete.

Or:

8b. Insertion of the femoral stem into the canal without the use of cement.

9. Inserting cement inside the femoral head “shell” and inserting the prosthetic head into said “shell” with meticulous cooling until cement polymerization is complete.

10. Reduction of the stem trunion into the prosthetic head.

11. Closure of the capsule.

Other alternative surgical procedures may also be usefully employed. One example of such an alternative would involve the dislocation of the femoral head immediately following its surgical exposure. The head could then be removed from the body and subjected to essentially the same procedure as described hereinbefore. These various surgical approaches may be combined with the use of different types of long-bone prosthesis including: two-part prostheses, monoblock prostheses, bipolar prostheses and monoblock bipolar prostheses. The stages involved in the use of these types of prosthesis in conjunction with the different types of surgical procedure described in this section, are disclosed and defined hereinabove.

The following examples are provided for illustrative purposes and in order to more particularly explain and describe the present invention. The present invention, however, is not limited to the particular embodiments disclosed in these examples.

### EXAMPLE 1

### A Rough-Surfaced, Cartilage-Sparing Long Bone Prosthetic Head According to the Present Invention

In one particularly preferred embodiment of the long-bone prosthetic head of the present invention, the rough outer surface is provided by the presence of slots or grooves cut into said surface. FIGS. 5 to 7 illustrate a typical femoral head endoprosthesis of the present invention, in which the surface roughness is provided by the presence of said slots and grooves.

Referring now to FIG. 5, it will be noted that the unpolished stainless steel prosthetic femoral head depicted therein (shown generally as 50) while generally spherical in shape, possesses a flattened, truncated base 51 which contains a circular opening 52 for receiving the proximal end of an appropriately-sized endoprosthetic femoral stem. In the embodiment of the femoral head shown in this figure, the geometric center of femoral head 50 is situated approximately 11 mm above flattened base 51. The external diameter of the essentially-spherical head 50 shown in this example is 32 mm.

The prosthetic femoral head depicted in this figure is characterized by the presence of two distinct types of surface feature. Firstly, there is a series of six, circumferentially-disposed grooves 53, the most inferior (distal) of which is situated 4.6 mm below a line defining the “equator” of the essentially spherical head (i.e. the horizontally disposed circumferential line of greatest length, when the prosthetic head is placed vertically such that the flattened base 51 is situated inferiorly). The circumferential groove situated immediately adjacent and superior to the above-described groove is situated at a distance of 1 mm below said equatorial line. The remaining four circumferential grooves are situated above said equatorial line and are separated therefrom (in order from below to above) by the following distances: 2.6 mm, 6.1 mm, 9.3 mm and 12 mm. The angular separation between each circumferential groove and its nearest neighbor is five degrees. Each of said circumferential grooves 53 has a mean depth of 1 mm. The width of each groove at its outer end is 1.2 mm, while the width at the inner end thereof tapers to 0.8 mm.

The second type of distinct surface feature of the prosthetic head depicted in this figure is a vertically-disposed slot 54 (of which two such grooves are depicted in FIG. 5.). It will be appreciated from the figure that said slots are situated along imaginary longitudinal lines, and are of such a length such that they intersect the three most superior (proximal) circumferential grooves 53 at an angle of 90° C. Said vertically-disposed slots are formed by the use of 5 mm diameter drill that is offered to the prosthetic head at an angle of 34 degrees in relation to the geometric center of the spherical head. The upper end of each slot is located 9 mm from the superior (proximal) pole of the spherical head, while the lower end thereof is located 3.5 mm above the above-defined equatorial line. A total of four such vertically-disposed slots are present in the prosthetic head, as shown more clearly in FIG. 7, wherein said slots are indicated as 71. It will further be appreciated from this figure that said vertically-disposed slots are arranged equidistantly from each other.

FIG. 6 schematically depicts the same prosthetic head (shown generally as 60) as presented in FIGS. 5 and 7 in inferior view (i.e. with the distal surface uppermost). In this figure, the truncated base 61 is shown to be perforated by circular opening 62, the purpose of which is to accept and retain the prosthetic stem section (not shown). Said opening leads to an internal space that is essentially conical in shape, the external surface of said cone having a diameter of 14 mm and the internal base thereof having a diameter of 12 mm.

### EXAMPLE 2

### A Micro-Roughened and Cratered Cartilage-Sparing Long Bone Prosthetic Head According to the Present Invention

In a further particularly preferred embodiment of the invention, the long-bone endoprosthetic head is characterized by the presence of a plurality of facets or craters distributed over its outer surface. In addition, essentially the entire outer surface of the prosthetic head is micro-roughened.

FIG. 10 depicts a prosthetic head of this embodiment of the present invention, generally indicated as 10. As may be seen, the generally spherical shape of the head is interrupted on its inferior side by a flattened base region 14, said base region being perforated by an opening 16 leading into the internal cavity of the prosthesis.

As shown in FIG. 10, the generally spherical contour of the prosthetic head has been interrupted or modified by the presence of plurality of craters or facets 12 that are distributed across the outer head surface.

Without wishing to be bound by theory, it is believed that craters 12 are able to improve the stability of the prosthesis-cement attachment by virtue of transforming shear stresses in the enveloping cement into compressive stresses. During the process of manufacturing the craters, all surface contours are gently rounded, thereby minimizing the development of localized concentrations of stress forces.

Following creation of the craters or facets, as described hereinabove, the outer surface of the prosthetic head is micro-roughened by means of grit blasting.

FIG. 11 depicts the various layers of the cartilage-preserving prosthesis of this embodiment of the present invention. As shown in the exploded view in FIG. 11A, the prosthetic head 20 is covered by a layer of biocompatible polymethylmethacrylate (PMMA) cement 22 having a thickness of 2-5 mm. (Examples of other suitable cements that may be used include Palacos cement, Simplex, CMW and Cementech.) In many cases, the preferred cement is PMMA cement. The aforementioned cement layer is used to stably grasp the cartilage preserving prosthesis inside the bone-cartilage long-bone head shell. Conceptually, the head shell can be considered as consisting of two layers: a thin (approximately 1 mm deep) layer of residual cancellous bone 24 infiltrated by the cement layer, and an approximately 5 mm thick layer of cortical bone overlaid with articular cartilage 26. It is to be recognized, however, that in practice, the two outer most layers—the cancellous bone layer 24 and the cortical bone/cartilage layer 26—are present as a single head shell, following removal of most of the cancellous bone. The process of removing most of the cancellous bone from the long-bone head is achieved by reaming the cancellous bone with a high or low speed burr or any other conventional acetabular reamer.

The complete prosthetic head-cement-bone/cartilage assembly is shown (in exterior view) in FIG. 11B.

FIG. 12 is an in situ cut-away view of the prosthetic head of this preferred embodiment of the present invention 30 after it has been cemented to a femoral head shell. As shown in the figure, the prosthetic head, with the plurality of craters 35 on its surface, is attached to the femoral head shell (consisting of a residual amount of cancellous bone 37 and cortical bone with its overlay of articular cartilage 38) with a layer of cement 36. The natural interface between the two anatomical articular surfaces (the femoral head cartilage 38 and the acetabular cavity cartilage 39) is thus preserved. The external entrance to the prosthetic head's interior cavity 34 (located in the centre of flattened inferior surface 32) will then receive a stem trunion (not shown), which in turn will be inserted and bonded into a pre-prepared femoral canal.

While specific embodiments of the invention have been described for the purpose of illustration, it will be understood that the invention may be carried out in practice by skilled persons with many modifications, variations and adaptations, without departing from its spirit or exceeding the scope of the claims.

1. A long-bone endoprosthesis comprising an essentially spherical head region that is connected to a narrowed stem section, wherein the head region is characterized in having a roughened outer surface.

2. The endoprosthesis according to claim 1, wherein the roughened prosthetic head outer surface has an arithmetic average roughness (R_{α}) value in the range of 0.05 μm to 500 μm.

3. The endoprosthesis according to claim 2, wherein the roughened prosthetic head surface has an arithmetic average roughness (R_{α}) value in the range of 40 μm to 200 μm.

4. The endoprosthesis according to claim 3, wherein the roughened prosthetic head surface has an arithmetic average roughness (R_{α}) value of 50 μm.

5. The endoprosthesis according to claim 1, wherein the roughened prosthetic head outer surface is a surface comprising one or more surface features selected from the group consisting of indentations, ridges, slots, grooves, pores, dimples and protuberances.

6. The endoprosthesis according to claim 5, wherein the surface features have an average depth or height in the range of 0.05 μm to 5000 μm.

7. The endoprosthesis according to claim 6, wherein the surface features have an average depth or height in the range of 400 μm to 2000 μm.

8. The endoprosthesis according to claim 7, wherein the surface features have an average depth or height of 1000 μm.

9. The endoprosthesis according to claim 5, wherein the roughened prosthetic head surface is a surface fitted with one or more grooves, pores and/or slots.

10. The endoprosthesis according to claim 9, wherein the grooves, pores and/or slots have an average depth of between 1 μm and the maximum thickness of the prosthetic head.

11. The endoprosthesis according to claim 1, wherein the long-bone is the femur.

12. The endoprosthesis according to claim 1, wherein the long-bone is the humerus.

13. The endoprosthesis according claim 1, wherein the head and neck regions are constructed as two separate modular units.

14. The endoprosthesis according to claim 13, wherein the head region comprises a bipolar head prosthesis.

15. The endoprosthesis according to claim 1, wherein the head and neck regions are constructed as a one-piece, monoblock unit.

16. A long-bone endoprosthetic head comprising an essentially spherical portion, into the distal side of which is formed a recess such that a femoral stem endoprosthesis may be inserted therein and connected thereto, and wherein said endoprosthetic head is characterized by having a roughened outer surface.

17. The long-bone endoprosthesis head according to claim 16, wherein the roughened head surface has an arithmetic average roughness (R_{α}) value in the range of 0.05 μm to 500 μm.

18. The long-bone endoprosthesis head according to claim 17, wherein the roughened head surface has an arithmetic average roughness (R_{α}) value in the range of 40 μm to 200 μm.

19. The long-bone endoprosthesis head according to claim 18, wherein the roughened head surface has an arithmetic average roughness (R_{α}) value of 50 μm.

20. The long-bone endoprosthesis head according to claim 16, wherein the roughened head outer surface is a surface comprising one or more surface features selected from the group consisting of indentations, ridges, slots, grooves, pores, dimples and protuberances.

21. The long-bone endoprosthesis head according to claim 20, wherein the surface features have an average depth or height in the range of 0.05 μm to 5000 μm.

22. The long-bone endoprosthesis head according to claim 21, wherein the surface features have an average depth or height in the range of 400 μm to 2000 μm.

23. The long-bone endoprosthesis head according to claim 22, wherein the surface features have an average depth of 1000 μm.

24. The long-bone endoprosthesis head according to claim 20, wherein the roughened prosthetic head outer surface is a surface fitted with one or more grooves, pores and/or slots.

25. The long-bone endoprosthesis head according to claim 24, wherein the grooves, pores and/or slots have an average depth of between 1 μm and the maximum thickness of the prosthetic head.

26. The long-bone endoprosthesis head according to claim 16, wherein said endoprosthesis head is a bipolar prosthetic head.

27. The long-bone endoprosthesis head according to claim 16, wherein the long-bone is the femur.

28. The long-bone endoprosthesis head according to claim 16, wherein the long-bone is the humerus.

29. A long-bone endoprosthesis having an essentially spherical head characterized by the presence of a plurality of crater-like depressions distributed over its outer surface, and further characterized in that substantially the entire outer surface has an average micro-roughness (R_{α}) in the range of 1 to 1000 micrometers.

30. The long-bone endoprosthesis according to claim 1, wherein the micro-roughness value is in the range of 30 to 70 micrometers.

31. The long-bone endoprosthesis according to claim 1, wherein the crater-like depressions are distributed across essentially the entire outer surface of the endoprosthetic head, and wherein the maximum depth of each depression is in the range of 0.2 to 3 mm and the diameter of each depression is in the range of 3 to 20 mm.

32. An endoprosthesis system comprising a long-bone endoprosthesis head according to claim 16, and a femoral stem endoprosthesis that may be connected thereto.

33. A method for treating fractures of the neck region of a long-bone in a patient in need of such treatment, wherein said method comprises the steps of:
a) removing most or all of the cancellous bone from the head of said long-bone, thereby forming a long-bone head shell;
b) preparing the long bone canal to accept a prosthetic stem;
c) inserting, and optionally cementing said prosthetic stem into said long-bone canal;
d) inserting and cementing a long-bone prosthetic head into said long-bone head shell; and
e) reduction of the stem region into the recessed region of said prosthetic head.

34. A method for treating fractures of the neck region of a long-bone in a patient in need of such treatment, wherein said method comprises the steps of:
a) removing the native long-bone head from its natural location in the body, by the use of a safe technique that does not cause injury to the cartilage or subchondral bone;
b) preparing the head shell outside the body on the operative tray;
c) preparing the long bone canal to accept a prosthetic stem section;
d) inserting the prosthetic stem section with or without the use of cement;
e) cementing the prosthetic head section to the shell formed in step (b);
f) reduction of the prepared prosthetic head together with the attached head shell into the joint of the patient being treated, and connection of the distal portion of said prosthetic head to said prosthetic stem section.

35. A method for treating fractures of the neck region of a long-bone in a patient in need of such treatment, wherein said method comprises the steps of:
a) removing the native long-bone head from its natural location in the body, by the use of a safe technique that does not cause injury to the cartilage or subchondral bone;
b) preparing the head shell outside the body on the operative tray;
c) preparing the long bone canal to accept the stem section of a monoblock prosthesis;
d) inserting said monoblock prosthesis stem section into said long bone canal with or without the use of cement;
e) cementing the head section of said monoblock prosthesis to the shell formed in step (b);
f) reduction of the prepared monoblock prosthesis head together with the attached head shell into the joint of the patient being treated.

36. A method for treating fractures of the neck region of a long-bone in a patient in need of such treatment, wherein said method comprises the steps of:
a) removing most or all of the cancellous bone from the head of said long-bone, thereby forming a long-bone head shell;
b) preparing the long bone canal to accept a prosthetic stem;
c) inserting, and optionally cementing said prosthetic stem into said long-bone canal;
d) cementing the outer head of a bipolar prosthesis to the shell formed in step (a);
e) placing prosthetic stem section trunion into the inner cavity of the inner prosthetic head;
f) inserting the inner prosthetic head into the inner cavity of the outer prosthetic head;
g) locking the bipolar head assembly by means of closing the locking ring.

37. A method for treating fractures of the neck region of a long-bone in a patient in need of such treatment, wherein said method comprises the steps of:
a) removing the native long-bone head from its natural location in the body, by the use of a safe technique that does not cause injury to the cartilage or subchondral bone;
b) preparing the head shell outside the body on the operative tray;
c) preparing the long bone canal to accept a prosthetic stem section;
d) inserting the prosthetic stem section with or without the use of cement;
e) cementing the outer head of a bipolar prosthesis to the shell formed in step (b);
f) placing prosthetic stem section trunion into the inner cavity of the inner prosthetic head;
g) inserting the inner prosthetic head into the inner cavity of the outer prosthetic head;
h) locking the bipolar head assembly by means of closing the locking ring;
i) reduction of the prepared prosthetic head together with the attached head shell into the joint of the patient being treated.

38. A method for treating fractures of the neck region of a long-bone in a patient in need of such treatment, wherein said method comprises the steps of:
a) removing the native long-bone head from its natural location in the body, by the use of a safe technique that does not cause injury to the cartilage or subchondral bone;
b) preparing the head shell outside the body on the operative tray;
c) preparing the long bone canal to accept the stem-inner section of a monoblock bipolar prosthesis;
d) inserting the prosthetic stem section of the stem-inner head bipolar monoblock bipolar prosthesis, with or without the use of cement;
e) cementing the bipolar prosthetic outer head section to the shell formed in step (b);
f) reduction of the prepared bipolar prosthetic outer head together with the attached head shell into the joint of the patient being treated;
g) inserting the bipolar inner head into the internal cavity of the bipolar outer head;
h) locking the bipolar head assembly by means of closing the locking ring.

39. A method for treating fractures of the neck region of a long-bone in a patient in need of such treatment, wherein said method comprises the steps of:
a) removing the native long-bone head from its natural location in the body, by the use of a safe technique that does not cause injury to the cartilage or subchondral bone;
b) preparing the head shell outside the body on the operative tray;
c) preparing the long bone canal to accept the stem-inner section of a monoblock bipolar prosthesis;
d) inserting the prosthetic stem section of the stem-inner head bipolar monoblock bipolar prosthesis, with or without the use of cement;
e) inserting the bipolar inner head into the internal cavity of the bipolar outer head;
f) locking the bipolar head assembly by means of closing the locking ring;
g) cementing the shell formed in step (b) onto the bipolar prosthetic outer head section;
h) reduction of the prepared bipolar prosthetic outer head together with the attached bipolar prosthesis into the joint of the patient being treated.

40. A method for treating fractures of the neck region of a long-bone in a patient in need of such treatment, wherein said method comprises the steps of:
a) removing most or all of the cancellous bone from the head of said long-bone, thereby forming a long-bone head shell;
b) preparing the long bone canal to accept the stem-inner head section of a monoblock bipolar prosthesis;
c) inserting, the prosthetic stem section of the stem-inner head bipolar monoblock bipolar prosthesis, with or without the use of cement;
d) inserting the bipolar inner head into the internal cavity of the bipolar outer head;
e) locking the bipolar head assembly by means of closing the locking ring;
f) cementing the bipolar prosthetic outer head section to the shell formed in step (b);

41. The method according to claim 33, wherein the long-bone is the femur.

42. The method according to claim 33, wherein the long-bone is the humerus.
